(19) United States
(12) Patent Application Publication
(10) Pub. No.: US 2009/0322513 A1
(43) Pub. Date: Dec. 31, 2009

(54) Medical emergency alert system and method

(76) Inventors: Franklin Dun-Jen Hwang, Glendora, CA (US); Ching Ching Huang, Glendora, CA (US)

Correspondence Address: Franklin D. Hwang, 949 E. Plymouth Street, Glendora, CA, 91940, US

(21) Appl. No.: 12/456,057

(22) Filed: Jun. 11, 2009

Related U.S. Application Data
(60) Provisional application No. 61/133,544, filed on Jun. 27, 2008.

(51) Int. Cl. G08B 23/00; G08B 1/08; A61B 5/00

(52) U.S. Cl. 340/539.12; 340/539.13; 340/573.1; 600/301; 455/404.2

(57) ABSTRACT

A medical emergency reporting system and methodology that utilize a wearable monitoring device to continuously monitor key physiological parameters of a person, and when measurements exceed programmed threshold levels, it will automatically issue a medical emergency alert along with location information to a remote monitoring center via a wireless network and the Internet for immediate local response. This system will also provide manual emergency alert activation, continuous updates with key physiological measurements to the emergency response personnel along with the medical history of the subject as well as redundancy in emergency alert reporting and malfunction diagnosis to assure ultimate accuracy, immediacy and reliability for the person that requires medical assistance.

### RELATED APPLICATIONS

This utility application claims the benefit of U.S. Provisional Patent Application No. 61/133,544 filed on Jun. 27, 2008, the non-provisional patent application Ser. Nos. 12/217,415 filed on Jul. 3, 2008 and 12/383,536 filed on Mar. 25, 2009.

### FIELD OF THE INVENTION

This invention relates to a system and method of measuring and reporting the physiological parameters of a person on a continuous basis. An emergency alert will be issued to a central monitoring station via wireless network and the Internet when the monitored parameter(s) becomes abnormal. The monitoring personnel, in turn, will notify an emergency response team local to the person issuing such alert to respond to the emergency along with the location and medical history and the latest physiological measurements of said person.

This automated medical emergency alert system can also be activated manually by the person when circumstance dictates, such as a fall or a criminal act being committed on him/her. Furthermore, the wireless network permits freedom of movement for the person and does not limit him/her to the place of domicile.

### BACKGROUND OF THE INVENTION

Emergency health crises that require immediate attention have been a difficult problem to address regardless of the age of the person that is encountering a medical emergency situation. There are panic-button type devices that interlink the person depressing the button to the emergency response team via landline or mobile telephone. However, if a person is disabled during a sudden health crisis, such as in a heart attack, a stroke or a serious fall situation, the panic-button type devices become useless. Furthermore, if the person is able to press the button, he/she must be within the effective wireless transmission distance to a device that dials the telephone to report the emergency, and no vital information on the person's status, like heart rate, blood pressure, breathing difficulty, etc. will be available for transmission to the response team. Consequently, the existing emergency reporting devices have only limited usefulness for the aging people and for those suffer special medical problems like Alzheimer's or dementia.

Since 1950, the number of persons older than 65 years of age in the United States has grown nearly three times from 12.2 million to 36.3 million in 2004, and it is projected to reach 86.7 million by 2050. Due to the improvement of healthcare and medicine, seniors now live longer and stay much more active. More and more retired people prefer to live by themselves while require less and less nursing care support. Consequently, many retirement communities have sprung up where seniors can live with independence while many organized community activities and supports are still available to them, if required. For those people and their grown children, the availability of a quick response to any health emergency is vital for peace of mind to maintain their independent way of living.

As a necessary condition to this trend of living independently, freedom of movement must not be restricted by a medical emergency monitoring system, nor should a system be of hindrance to one's daily activities. Furthermore, such a monitoring system must have an adjustable emergency alert level throughout a day for different levels of activity as well as the capability to determine the location of the person in need of assistance accurately and immediately to be effective and relevant. Global position systems (GPS) allow the location of a special receiver to be pin-pointed when signals from multiple GPS satellites are received by it. This approach's weak points are the inability to determine a position when not receiving signals from more than one satellite (due to shielding by buildings or geographic features or improper antenna orientation) as well as more power consuming electronics. Another method is using fixed cell phone towers to locate a mobile phone (cell tower triangulation) provided signals can be received by those towers. A third method is using a fixed array of radio frequency transceivers (integrated receivers and transmitters) distributed over a specific area to form a wireless local area network (WLAN) to relay signals wirelessly from a mobile device to a specific point, such as to a monitoring center or a gateway to the Internet, which in turn transmits the signal to a remote monitoring center. By using either time of arrival or signal strength of a mobile device reaching a distributed transceiver, the location of the mobile device can be determined. However, this third method based on WLAN must be able to identify the correct person in need of assistance when multiple persons are transmitting at the same time.

The invention outlined herein provides the solution of reporting any medical emergency situation while remedying all the shortcomings of the panic-button type of devices by continuously monitoring health parameters such as pulse rate, EKG, blood pressure, breathing rate, body temperature, etc. of the subject without interfering with a person's daily activities. It then reports the measurements and position of the person every few seconds to the central monitoring station via links in a wireless network. When there is a sudden abnormal change on the person's health parameters, it will automatically alert the central monitoring station to send a local response team to the person immediately.

Not only does this invention provide constant monitoring of one's health parameters, but it also allows complete freedom of movement of a person being monitored through one or more wireless networks. It can locate a person within its network as well as outside of it, and it can forewarn the response team on the type of health emergency encountered as well as provide continuous updates on the physiological conditions of the subject (a particularly critical set of information for proper treatment of heart attack and stroke victims). Furthermore, it carries the redundancy of emergency reporting through overlapping links in the WLAN along with redundancy in determining a person's location and the capability to determine the nature of system or device failure (subject not wearing the physiological sensor device, device failure, power drain on the device or out of the coverage area, etc.).

This invention utilizes: (1) physiological parameter measurements as a key to detect any medical emergency conditions and will issue alerts automatically, (2) wireless network to provide freedom of movement for the subjects under monitoring while furnishing accurate location determination without complicated and bulky equipment and high power consumption concern, (3) a remote monitoring center to activate local emergency response to the subject in need and providing up-to-moment critical physiological parameter measurements and medical history to the responding medical personnel, and (4) a methodology that furnish accuracy and redundancy in reporting emergency as well as diagnosis on any malfunction of the system to assure continuous and accurate monitoring and reporting.

The search and differentiation with prior arts can be divided into four categories: (1) panic button type activation of emergency notification through landline phone or cell phone to either 911 or a specific response entity, (2) personal emergency reporting systems with location determination capability, (3) out-patient monitoring systems, and (4) medical alert systems.

The first category and begins with U.S. Pat. No. 4,057,790 (granted on Nov. 8, 1977 to Fleming, et al.), it described an invention of a switch/button activated portable radio transmitting device to alert monitoring personnel to an emergency situation. Variations of this type of panic button approach can be seen in U.S. Pat. No. 4,829,285 (May 9, 1989, Brand, et al.—adding a tilt switch to detect a fall and automated emergency alert issuing), both U.S. Pat. No. 5,045,839 (Sep. 3, 1991, Ellis, et al.) and U.S. Pat. No. 6,201,476 (Mar. 13, 2001, Depeursinge, et al.—using multi-axis motion detector as well as proximity to a base station to automate emergency alerting and location determination), U.S. Pat. No. 5,929,761 (Jul. 27, 1999, Van der Laan, et al.—adding an audio communication channel between the person activating emergency alert and the monitoring entity), U.S. Pat. No. 6,044,257, U.S. Pat. No. 6,636,732, and U.S. Pat. No. 7,251,471 (Mar. 28, 2000, Oct. 21, 2003, and Jul. 31, 2007, Boling, et al.—using cellular technology as a reporting conduit instead of landline phone network), and U.S. Pat. No. 7,394,385 (Jul. 1, 2008, Franco, Jr., et al.—adding mobile repeaters to extend the movement range of a person under monitoring). Also, commercially available systems, such as Life Alert™ (El Segundo, Calif.) and AlertOne (Williamsport, Pa.), use a panic button in the form of a pendant to wirelessly activate the base station to dial 911 or a special monitoring center via landline phone. All these prior arts and commercial products depend mostly on manual activation of the panic button (except those with tilt switch or 3-axis motion detector that can sense a fall and issue alerts automatically). None provide physiological measurements as a mean to determine whether a medical emergency situation has arisen, and none use abnormal physiological measurements as an automatic trigger to issue emergency alert and obtain immediate response without the person's manual effort of depressing a panic button. The panic button approach that utilizes landline telephones to issue emergency alerts to a monitoring center restricts bearers to be within a specific distance of a base unit that dials the telephone; while approaches use cellular technology as communication conduits lack the means to specify the location of a bearer in need of emergency help without the person well enough to state where he/she is. Neither of these approaches provides redundancy in ascertaining that an emergency alert reaches the monitoring center, nor any warning and diagnosis of malfunctions on the system or devices within.

The personal emergency reporting systems with location determination capability are the second category of prior arts that deal with emergency reporting. The scope of this type of reporting and location identification can be represented by the U.S. patents like U.S. Pat. No. 6,028,514 (Feb. 22, 2000, Lemelson, et al.—using fixed geographic transmitter/receiver to locate and warn persons of dangers), U.S. Pat. No. 6,198,394 (Mar. 6, 2001, Jacobsen, et al.—using Global Positioning System (GPS) to locate a person and using wearable sensors to determine the physiological wellness of the subject for emergency response purpose), both U.S. Pat. No. 6,166,639 and U.S. Pat. No. 6,333,694 (issued on Dec. 26, 2000 and Dec. 25, 2001 to Pierce, et al.—using fixed location transmitter to activate sensors worn by a person within its range to indicate and report distress), U.S. Pat. No. 7,307,522 (Dec. 11, 2007, Dawson—using a manually activated mobile radio frequency transmitter to report an emergency as well as to trigger a close-by location indicator to transmit its location information corresponding to the emergency report), and U.S. Pat. No. 7,423,528 (Sep. 9, 2008, Otto—using two way wireless communication to verbally report emergency and location). Virtually, all the prior arts of this category require manual activation of an emergency alert instead of automatic activation by measured physiological parameters. There are no continuous updates on the physiological measurements to facilitate a proper medical response. Furthermore, all of them use location determination methods without dealing with failure of these methods (such as unable to acquire multiple GPS satellite signal to determine a location, location confusion caused by multiple RF transmitters transmitting at the same time to an RF access points of WLAN, etc). Again, there is no redundancy in making sure that an emergency alert can reach the monitoring center, nor any warning and diagnosis of malfunctions on the system.

The third category of the prior arts is the out-patient status monitoring as represented by U.S. Pat. No. 3,972,320 (Aug. 3, 1976, Kalman), U.S. Pat. No. 4,129,125 (Dec. 12, 1978, Lester, et al), U.S. Pat. No. 4,712,562 (Dec. 15, 1987, Ohayon, et al.), U.S. Pat. No. 5,558,638 (Sep. 24, 1996, Evers, et al.) U.S. Pat. No. 5,919,141 (Jul. 6, 1999, Money, et al.), U.S. Pat. No. 6,160,478 (Dec. 12, 2000, Jacobsen, et al.), U.S. Pat. No. 6,287,252 (Sep. 11, 2001), U.S. Pat. No. 6,315,719 (Nov. 13, 2001, Rode, et al.), U.S. Pat. No. 6,402,691 (Jun. 11, 2002, Peddicord, et al.), U.S. Pat. No. 6,384,728 (May 7, 2002, Kanor, et al.), U.S. Pat. No. 6,412,471 (Jul. 9, 2002, Kumar, et al.), U.S. Pat. No. 6,454,708 (Sep. 24, 2002, Ferguson, et al), U.S. Pat. No. 6,616,606 (Sep. 9, 2003, Peterson, et al.), U.S. Pat. No. 6,746,398 (Jun. 8, 2004, Hervy, et al.), both U.S. Pat. No. 6,988,989 and U.S. Pat. No. 7,390,299 (Jan. 24, 2006 and Jun. 24, 2008 by Weiner, et al.), both U.S. Pat. No. 7,088,233 and U.S. Pat. No. 7,138,902 (Aug. 8, 2006 and Nov. 21, 2006, Menard), U.S. Pat. No. 7,129,836 (Oct. 31, 2006, Lawson, et al.), U.S. Pat. No. 7,161,484 (Jan. 9, 2007, Tsoukalis), U.S. Pat. No. 7,215,991 (May 8, 2007, Besson, et al.), U.S. Pat. No. 7,407,484 (Aug. 5, 2008, Korman), and U.S. Pat. No. 7,423,526 (Sep. 9, 2008, Despotis). All of these prior arts report physiological measurements of an out-patient to a processor at a fixed location via wired or wireless mean; the subject is tethered to a processing unit, which, in turn, is itself physically connected to a communication network, thus limiting the area mobility of the subject. The transmitted physiological data is to be evaluated by his/her caregivers and not as criteria for emergency reporting. Furthermore, these prior arts principally measure a single patient and transmit the measured data to the targeted caregivers; consequently, no methodologies are devised to deal with identification of multiple patients and matching their physiological data accurately.

The fourth category of prior arts that is closest to the present invention can be grouped under the term “medical alert reporting”. All of them contain the functionalities of monitoring one or more physiological parameter of a subject and automatically reporting any abnormality as an emergency to a monitoring center or to a preset list of first responders, including 911. However, none deals with accurate matching of a person's physiological measurements with one's identity in an environment of multiple persons transmitting at the same time. Also, none provide a fool-proof method of determining the location of the subject in need of medical emergency response when freedom of movement is part of the system's essential features. None provide a continuous update of the physiological measurements during the emergency reporting and time period before the arrival of a response team to give these responders relevant data to anticipate proper recourses to take. Also, none of these prior arts provided redundancy in ascertaining that an emergency alert can reach the monitoring center, nor any warning and diagnosis of malfunctions on the system as well as its components. Most important of all, none of the prior arts can furnish an adjustable medical alert level due to change of physical conditions of a subject under monitoring throughout a day or due to anticipated action, such as change of heart rate or blood pressure caused by new medication to avoid false alarm. Differentiations on key prior arts are detailed below.

U.S. Pat. No. 5,288,449 (Jul. 20, 1993, Christ, et al.)—This invention deals primarily with out-patient having a cardiac emergency. It ties the patient to the sensor/communication unit at a fixed location. Also, this system does not provide continuous updates on the physiological conditions of the person in need of assistance. U.S. Pat. No. 5,335,664 (Aug. 9, 1994, Nagashima) has identical limitations as the patent described above. While U.S. Pat. No. 5,754,111 (May 19, 1998, Garcia) provides a medical alert system to issue alerts to redundant EMS upon receiving a health status alert from a non-specified monitoring source. This patent does not deal with how medical alert status is first determined, nor does it deal with location data of the person in stress.

Both U.S. Pat. No. 5,898,367 (Apr. 27, 1999, Berube) and U.S. Pat. No. 6,624,754 (Sep. 23, 2003, Hoffman, et al.) are personal security systems furnishing manual emergency reporting as well as position determination via wireless means. Both of these prior arts do not employ physiological sensors to measure the medical condition of the subject as criteria for issuing alert automatically. Also, they employ GPS, LORAN-C, GLONASS or ELT for location determination, which requires far more complicated electronic equipment and high power consumption than the present invention uses.

U.S. Pat. No. 6,198,394 (Mar. 6, 2001, Jacobsen, et al.)—This invention uses GPS and physiological sensors to detect the position and physical wellness of soldiers in the battlefield. An alert will be issued to commanders and medics for quick response. It depends in significant physiological changes, such as large blood pressure drop due to wounds, to issue an alert while the present invention deals with changes from historical normal measurements of the person under monitoring. Jacobsen uses only GPS for location determination while the present invention uses a combination of signal strength measurements and historical movement data method through access points of a Wireless Local Area Network (WLAN), in addition of having a backup GPS method. This ensures accuracy and reliability in location determination. Furthermore, Jacobsen, et al. can not provide adjustable threshold for alert due to the extreme circumstances a soldier will typically experience in battlefield, while the present invention does present this capability for people with ordinary living routines. U.S. Pat. No. 5,874,897 (Feb. 23, 1999, Klempau, et al.) is very similar to U.S. Pat. No. 6,198,394 by using GPS for location determination. However, its inclusion of a computer, control unit, GPS receiver and emergency transmitter into a separate unit from the portable patient data unit limits the distance that the subject can be away from this base unit. Furthermore, the power consumption of this base unit dictates a physical connection to a power source for best operating time length instead of using batteries. Other key differentiation stated vs. U.S. Pat. No. 6,198,394 applies to this invention by Klempau, et al. as well.

U.S. Pat. No. 6,747,561 (Jun. 8, 2004, Reeves) describes a wearable device that stores the personal identity and medical record to facilitate treatment by EMS personnel in a medical emergency situation. However, this invention is not a medical monitoring or an emergency alert reporting system; it also does not provide location information to any specific monitoring center.

U.S. Pat. No. 7,154,398 (Dec. 26, 2006, Chen, et al.) presents a health monitoring and reporting system based on GPS for location determination and sensors to measure the health parameters of a subject. This system not only carries the shortcoming of using GPS for location determination (failure to provide accurate location data when signals from multiple satellites are absent, such as within a building, high power consumption and large physical package, etc.), but also provides no redundancy to insure emergency alert being received by the monitoring center or indication of system failure as the present invention does. Also, Chen's prior art does not provide an adjustable alert threshold to accommodate subjects with a variety of conditions and transient situations. U.S. Pat. Nos. 7,382,247 (Jun. 3, 2008, Welch, et al.), 7,405,653 (Jul. 29, 2008, Tice, et al.) and patent application number 20060008058 (Jan. 12, 2006, Dai, et al.) are similar to U.S. Pat. No. 7,154,398 except no location determination capability is included in these inventions. Consequently, these prior arts are only suitable to monitor a subject within a range of a fixed location, contrary to the key feature of freedom of movement for the subject presented by the current invention.

U.S. Pat. No. 7,221,928 (May 22, 2007, Laird, et al.)—This patent does use a combination of GPS and Wireless Local Area Network (WLAN) for locating a person carrying a specially adapted emergency reporting handset. However, there are no physiological sensors to issue an automatic emergency alert; the person must carry the special handset instead of wearing the sensor/transceiver on the wrist or arm; there is no provision for the lack of signals from multiple GPS satellites and no fool-proof identification of the subject when there is interference from other persons' handsets when employing its stated WLAN method. U.S. Pat. Nos. 7,289,786 (Oct. 30, 2007, Krasner), 7,315,736 (Jan. 1, 2008, Jenkins) and 7,480,501 (Jan. 20, 2009, Petite) describe similar personal emergency communication systems based on cellular telephone technology, and the differentiation vs. Laird's invention mentioned above applies to these three prior arts.

U.S. Pat. No. 7,261,691 (Aug. 28, 2007, Asomani) describes a wearable device that reports abnormal and dangerous glucose levels of a subject to an emergency response center. The system also includes an unspecified geolocation system to provide the whereabouts of the subject. Since glucose level measurement must involve manually pricking one's finger or arm to obtain a blood sample, this system is not automatic or transparent to the subject in providing monitoring and issuing emergency alert. This prior art does not cover other key physiological measurements, such as pulse rate, EKG, blood pressure, breathing rate, body temperature, etc. to indicate abnormal medical conditions of the subject; nor it provides the adjustable alert level and redundancy that the current invention furnishes.

### SUMMARY OF THE INVENTION

This invention presents a system (hardware and operating software) and methodology to provide continuous monitoring of key physiological parameters of a person by means of wearable sensors for issuing automatic medical emergency alerts along with location information to a remote monitoring center via a wireless network and the Internet for immediate local response. This system will also provide manual emergency alert activation, continuous updates with key physiological measurements to the emergency response personnel along with the medical history of the subject as well as redundancy in emergency alert reporting and malfunction diagnosis to assure ultimate accuracy, immediacy and reliability for the person that requires medical assistance.

### BRIEF DESCRIPTION OF THE DRAWINGS

FIG. 1 illustrates a wrist watch version of a wearable monitoring device [1]; its components are: the enclosure for a rechargeable battery [2], the housing for digital processing and transceiver circuitry along with watch module [3], the plastic enclosure for the antenna [7], and a pulse rate sensing transducer as well as a temperature sensor indicated by [8] and [9].

FIG. 2 shows how other wireless sensors, EKG [11], blood pressure [10], etc. are attached on a human body and form a monitoring net with the wearable monitoring device [1].

FIG. 3 illustrates how an electromagnetic battery charger [12] is used to recharge the battery [2] within a wearable monitoring device without any requirement of alignment to electrical contacts.

The block diagram in FIG. 4 describes the design of the monitoring device [1] and interconnection with the various sensors, such as EKG [11] and blood pressure [10] via wireless approach, that complete the monitoring and processing of the health parameters of a person as well as transmitting the data to a remote monitoring center through a relaying transceiver device and the Internet.

FIG. 5 outlines in block diagram how a monitoring device [1] is interconnected with first [13, 14, 15] and second tiers relaying transceivers [16] as well as an Internet gateway device [17] to reach the remote monitoring center.

FIG. 6 illustrates a monitoring network of distributed relay devices that allows the freedom of personal movement while still providing the report of any emergency, while

FIG. 7 shows a conceptual monitoring center with screens displaying reported emergency cases and the associated data for the monitoring personnel to act immediately.

FIG. 8 demonstrates the type of medical emergency alert flashed on a monitoring screen at the remote monitoring center. The data flashes red and highlights the relevant information for the monitoring personnel to respond immediately as well as provides the physiological measurements continuously to a local response team through the monitoring center.

FIG. 9 presents a normal health parameters data recorded by the remote monitoring center. The data will be archived at the remote monitoring center and it will not be displayed on any monitoring screen except when recalled by supervisory personnel or the subject's authorized physicians.

FIG. 10 shows how signal strength received by a first tier relaying transceiver will be used to determine the radius [21] where the subject can be located. By combining the signal strength measured by multiple relaying transceivers, a person's location can be pin pointed. Furthermore, the prior locations of this person determined by his/her prior transmissions (the resulting track as represented by [22] can add to the accuracy of locating the person.

### DETAILED DESCRIPTION OF THE INVENTION

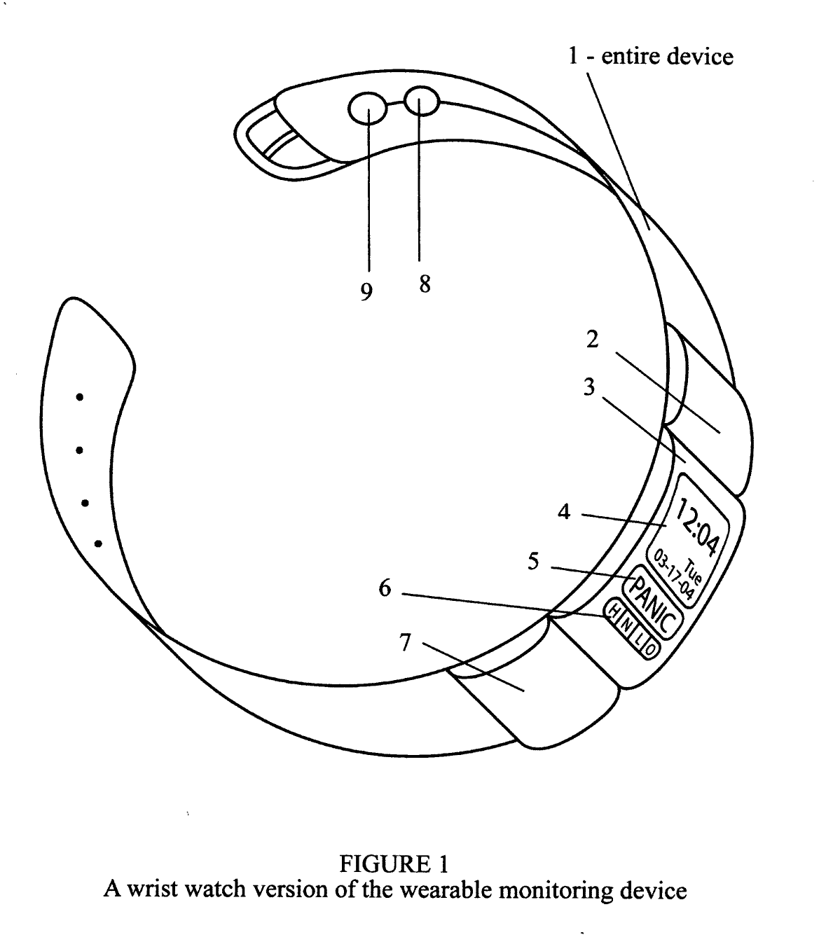

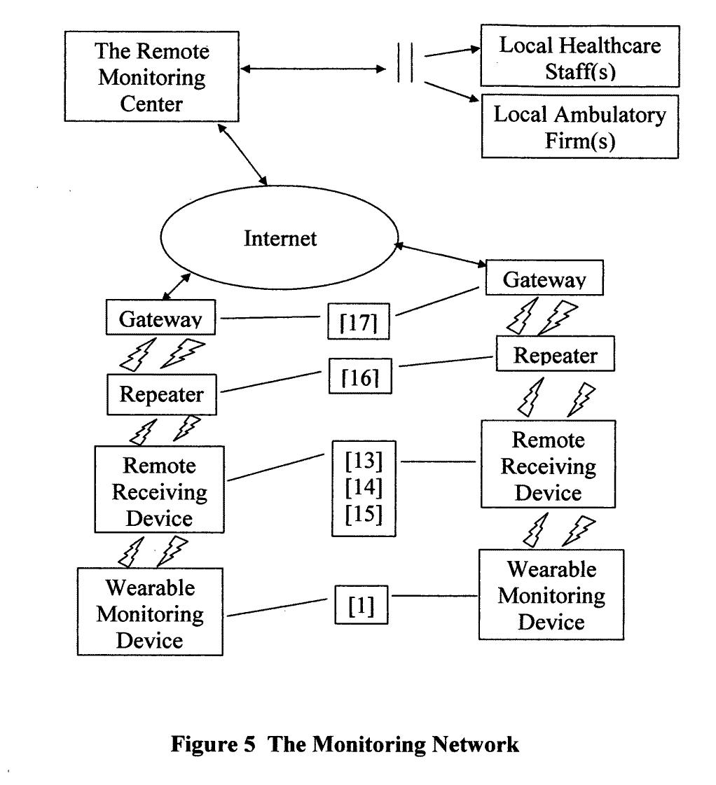

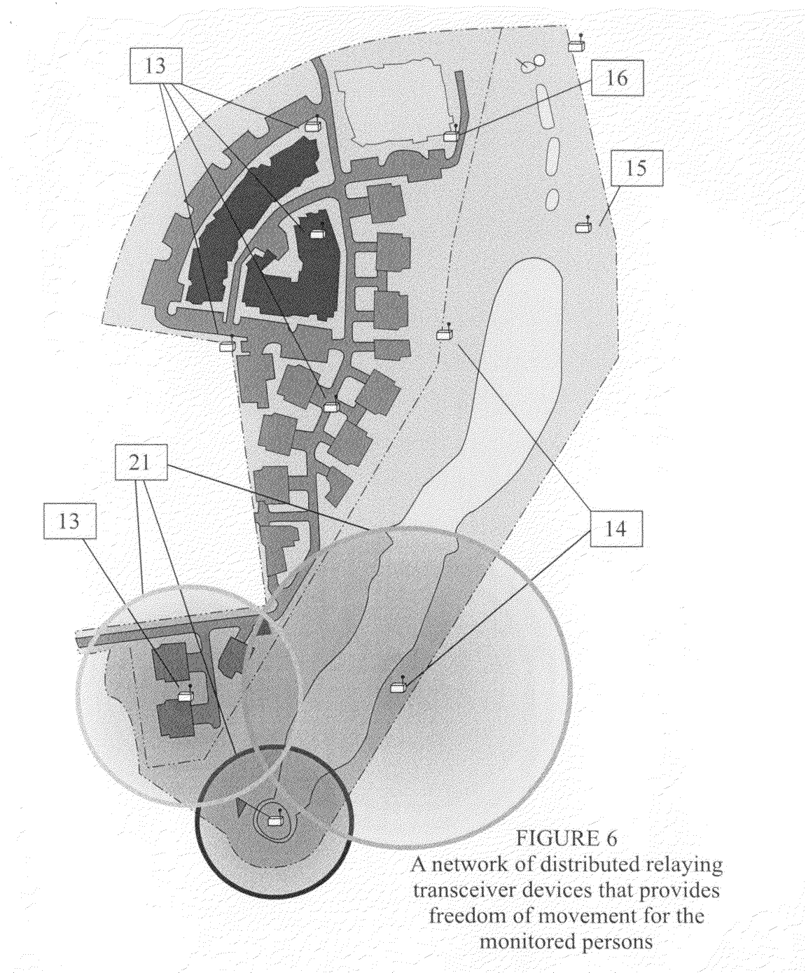

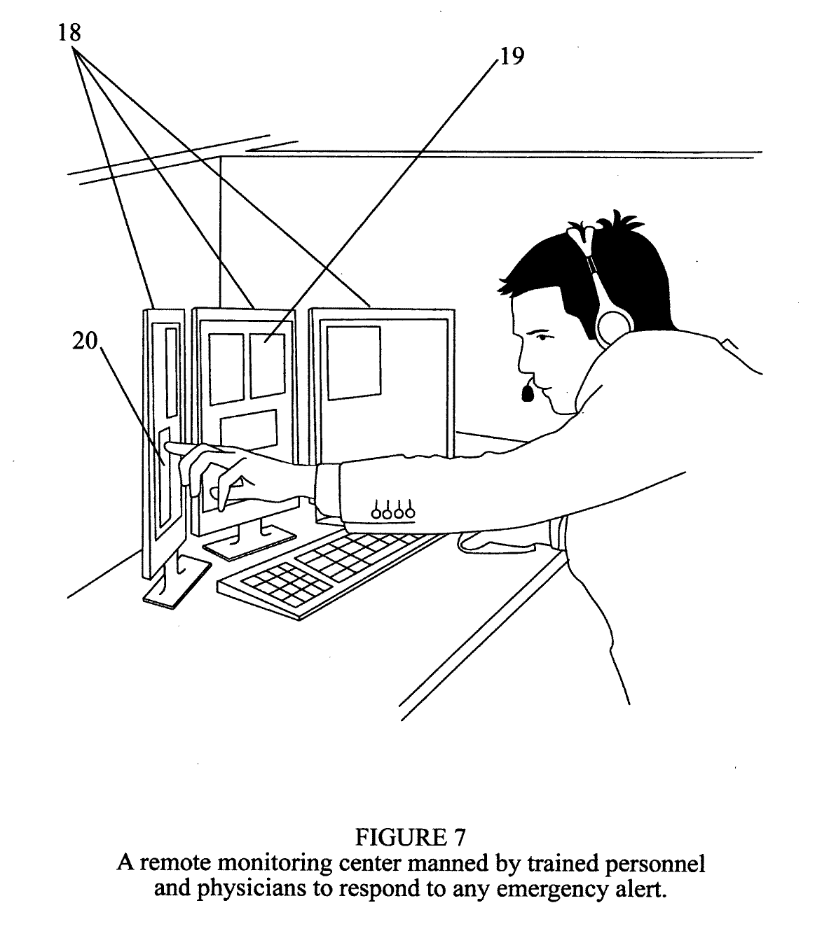

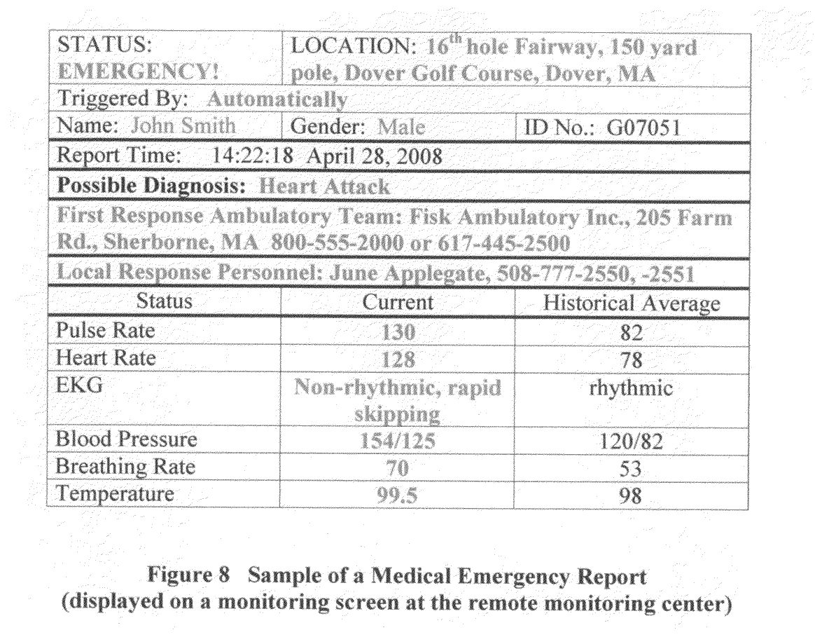

A wearable physiological measurement device, as shown in FIG. 1, is employed in this invention as the key of providing continuous monitoring of a person's health condition. This wearable monitoring device periodically and wirelessly transmits the measured results along with its unique identification code to a remote monitoring center for archiving. It will also automatically issue emergency or abnormality alerts when any measurement exceeds pre-programmed thresholds. A network of distributed transceivers, [13, 14, 15] in FIG. 6, will receive the wireless signals from any wearable monitoring devices within its coverage area and relay the signals along with each transceiver's own identification code and the information on the strength of signals it received from a wearable monitoring device (for location determination of the subject) to either a second tier relaying transceiver device, [16] in FIG. 6, or directly to an Internet gateway device [17] in FIG. 5 for transfer to the remote monitoring center. The remote monitoring center will archive any measurements corresponding to each person that is within the normal range, but it will flash any abnormality or emergency alert on a monitoring screen with associated support information, as illustrated in FIGS. 7 and 8, for the monitoring personnel to respond immediately. A local ambulatory firm and/or resident response personnel will be integral to this setup. They will be notified via phone or text message on the name and position of the person in need of assistance, type of emergency (probable diagnosis), medical data and history of the person as well as the latest physiological measurements.

A practical application is envisioned below in conjunction with the drawings to describe this invention in details:

A network of relaying transceivers is distributed within each residential unit, hallways, elevators, dining halls, activity rooms, external walkways around buildings, tees, fairways and greens of adjacent golf courses as illustrated in FIG. 6. The callouts [13, 14 and 15] in FIG. 6 indicate the first tier relaying transceivers, which are tuned to receive the radio frequency transmitted by a wearable monitoring device [1] on a person, such as 2.4 GHz. With this type of network and overlapping relaying transceivers, a person subscribed to this medical emergency alert service will have the total freedom of movement anywhere within the network.

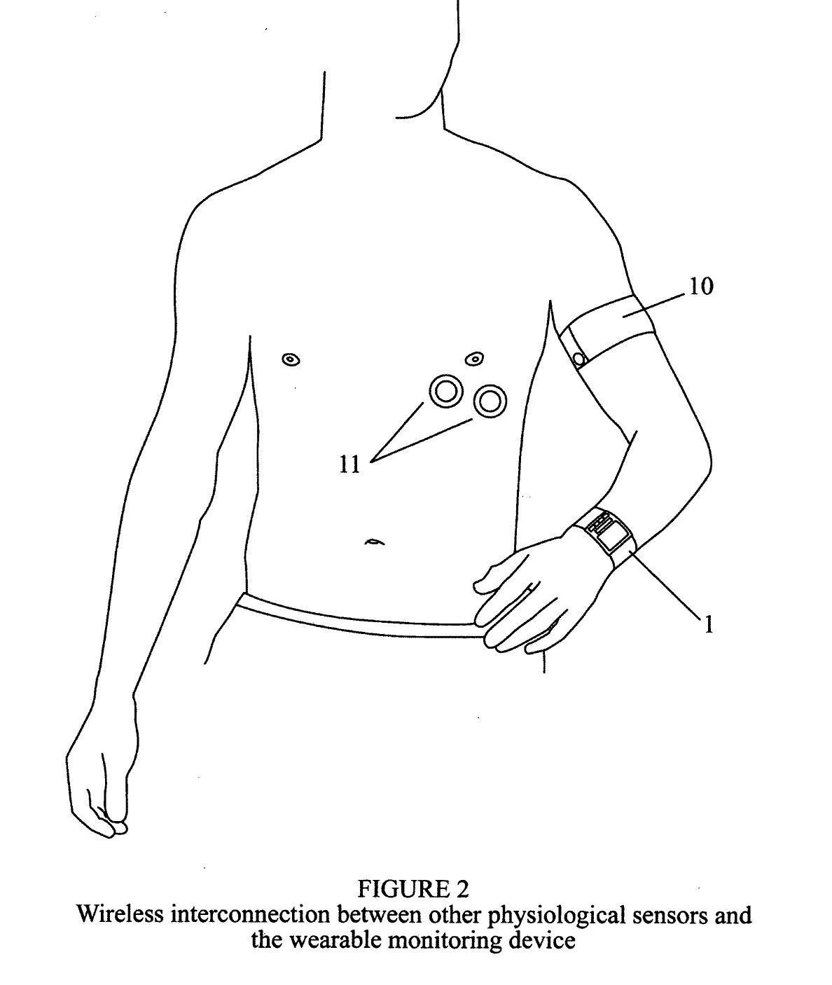

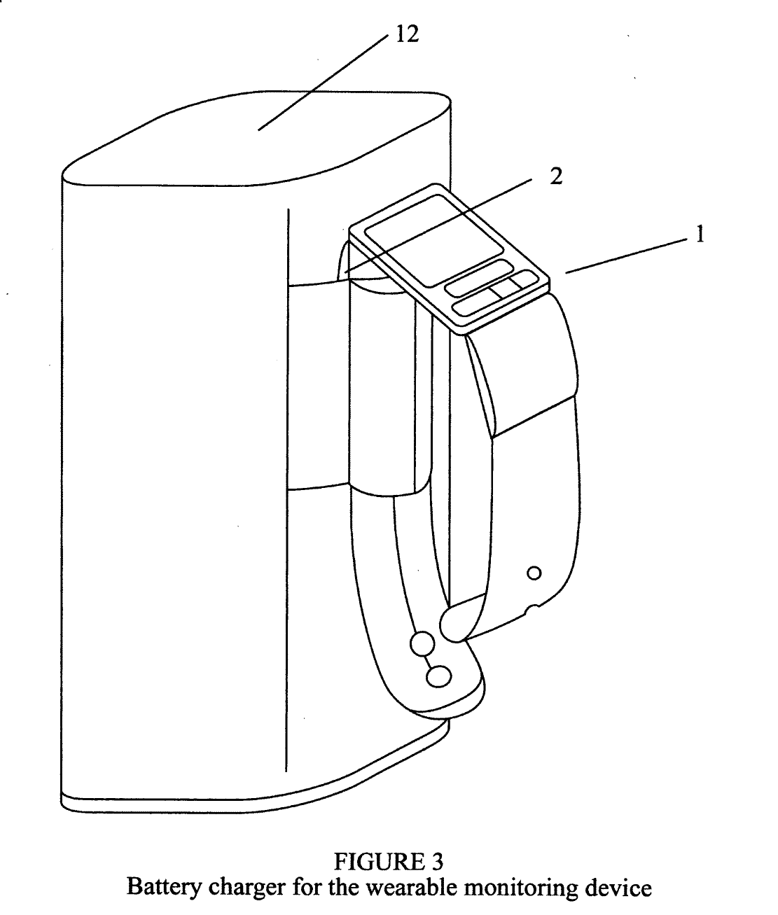

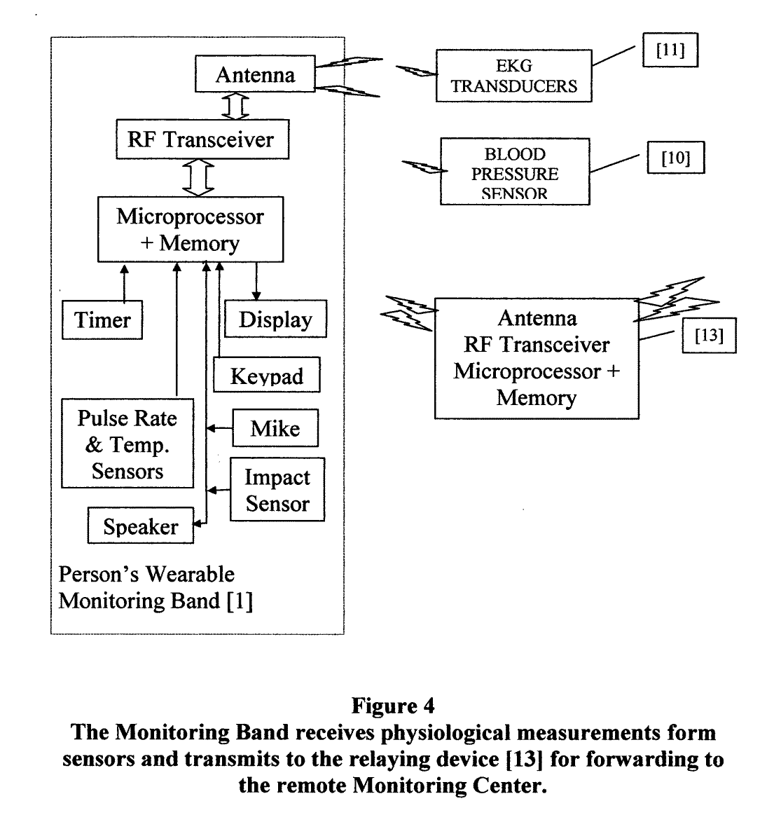

Each person will be assigned two wearable monitoring devices, [1] in FIG. 1, with each device containing the same identification code, and a battery charger [12] in FIG. 3. The pulse rate sensor [8], calibrated temperature sensor [9], EKG sensors [11] and/or blood pressure sensor [10] in FIGS. 1 and 2 will measure these physiological parameters periodically (such as at every 15- or 30-second interval) and compare the measurements with pre-programmed threshold levels to determine whether to issue an emergency or abnormal alert or normal signal string with the measured data. The monitoring device will also issue an immediate emergency alert when its internal impact sensor detects a fall or its membrane microphone detects a high amplitude sharp yell followed by moans. Upon issuing an emergency alert, it will perform and transmit the physiological measurement at faster repetition rate (such as every 5 seconds). Any signal transmission from a monitoring device will include its identification code. Also, it will not react to any received signal, if it does not include the identification code matching its identification code stored within. Therefore, its internal processor will not decipher any communication, verbal or text, from the remote monitoring center without its own identification code, thus eliminating interferences by messages to and from other subjects.

The triggering levels of an emergency alert for a monitoring device can first be programmed with a general set based on age, gender and normal medical conditions of the general population. These levels can vary according to the time of a day to accommodate the amount and types of activities that one undergoes. The physiological measurements (archived at the remote monitoring center) through a period of time will then be used to adjust the alert levels to fit the particular person by the data processor at the remote monitoring center. Furthermore, these levels can be adjusted by the subject under monitoring or by his/her physician to reflect change of physical conditions due to change of medication or treatment.

To ensure accuracy in matching reporting signal strings with a transmitting monitoring device by a relaying transceiver, the monitoring device will first transmit its identification code periodically (such as once every second) for a period of 10 to 15 seconds when it initiates a reporting sequence until one or more relay transceivers responds by issuing a “send” signal along with this monitoring device ID code. The monitoring device, then, will transmits its data string repeatedly in burst mode till all responding relaying transceivers each sends a “received” signal back (again including the ID code of the monitoring device). This hand-shaking method ensures that each reporting signal strings are received properly by one or more relaying transceiver, matched with the monitoring device ID code and forward to the remote monitoring center accurately. Furthermore, receiving and relaying by more than one relaying transceivers will provide the redundancy in ensuring reporting from a monitoring device will be received by the remote monitoring center.

The first tier relaying transceivers, [callouts 13, 14, 15] in FIG. 6, first compares the received signal strength with the five or more preset strength levels stored within to assign a strength index; it will then retransmit the received signal string along with the strength index and its own identification code (to constitute the key factors for the data processor at the remote monitoring center to calculate the location of the wearable monitoring device/person) at a different power level and frequency, such as 315 MHz, for long distance transmission to a second tier relaying transceiver [16] (which is tuned only to receive the transmission frequency of the first tier transceivers) or directly to an Internet gateway device [17]. Again, the second tier relaying transceiver and the Internet gateway device will conduct a similar hand-shake procedure as described in previous paragraph to ascertain the accuracy of the data received as well as matching with the source.

The Internet gateway device [17] will convert the received RF signals into TCP/IP protocol for transmission via the Internet to the remote monitoring center. Both the first and second tier relaying transceivers [13 through 16] and gateway devices [17] will provide the digitized two-way communication for this system. The interconnection among all the constituents of the system is illustrated in FIG. 5.

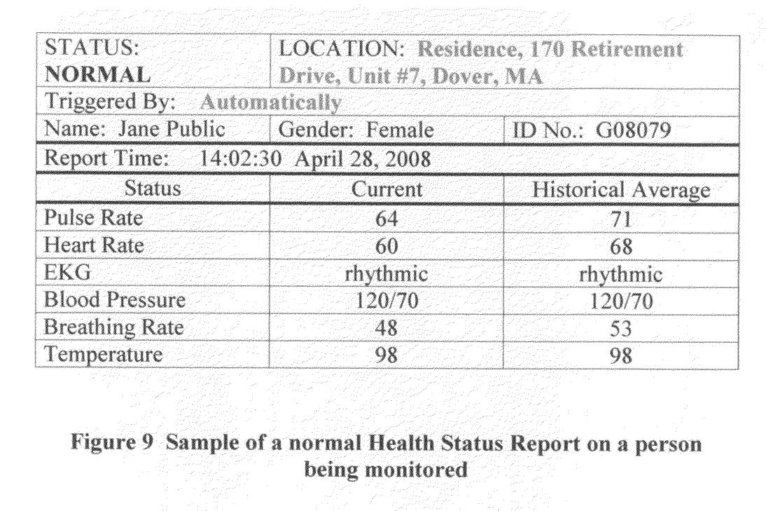

The remote monitoring center as shown in FIG. 7 will be manned 24 hours a day and seven days a week by trained personnel and some physicians. Each monitor screen [18] will be dedicated to a geographic area and display any emergency alert (in flashing red frame shown as [19] in FIGS. 7 and 8) and abnormality alert (in amber frame, shown as [20] in FIG. 7). Normal physiological measurement data of a person being monitored (as presented in FIG. 9) will be archived and not displayed. Upon receiving an emergency alert, an on-site physician can be consulted to make a preliminary diagnosis to properly prepare the local response team.

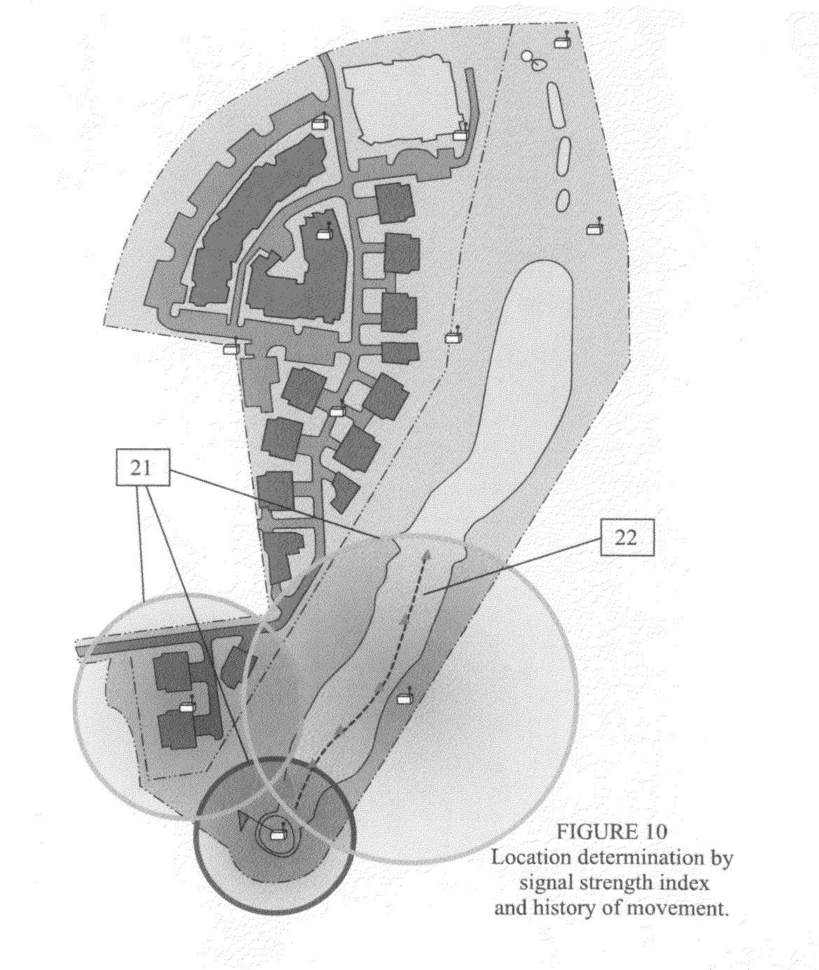

When a first tier relaying transceiver [13, 14, and 15] relays the signals from a wearable monitoring device, it will also transmit its own assigned unique identification codes and the signal strength index of the signals received. As illustrated in FIG. 10, the data processor of the remote monitoring center will be able to determine the location of the wearable monitoring device by the fixed locations of two or more relaying transceivers [13, 14, 15] in FIG. 10 and the size of the circles represented by the signal strength indices [21]. Furthermore, the movement track of the person, [22] in FIG. 10, recorded from previous signal transmissions is also plotted on the monitoring screen to provide complete accuracy (based on distance covered per unit of time and direction of travel) in determining the location of the person. Furthermore, a wearable monitoring device can have a GPS receiver added to provide a redundant location data to the remote monitoring center. Consequently, this invention not only provides medical emergency alerts with preliminary diagnosis, up-to-moment physiological measurement data and medical history of the person, but also accurate location of the person in need of assistance while allowing full freedom of movement to the person under monitoring.

One of the key features of this invention is the built-in redundancy of ensuring the system works and performs accurately. First, the rechargeable battery is designed to provide functional power for a few days of continuous operation along with low battery warnings to prompt recharge. Second, there is a redundant wearable monitoring device assigned to each person being monitored to give immediate replacement during battery charging or malfunction of the first monitoring device. Third, the network of distributed first tier relaying transceivers will provide overlapping coverage throughout a designated area to ensure each signal transmitted by a wearable monitoring device will have two or more relaying transceivers transmitting the signals to the remote monitoring center. Fourth, the data processor at the remote monitoring center can diagnose any malfunction of a wearable monitoring device and inform the wearer or the resident local response personnel, by two-way verbal or text communication, to remedy the situation. As an example, low battery level will trigger the monitoring device to warn the wearer as well as the remote monitoring center, thus initializing dual approaches to correct the problem. Another symptom that may arise is a signal string containing no physiological measurements can be diagnosed from previous signal strings. The center can deduce whether it is due to the person no longer wearing the monitoring device or sudden device/sensor malfunction. Again, through two-way communication or local response personnel, the problem can be quickly resolved. If there is no signal received from a particular monitoring device after two or more reporting cycles have elapsed, the center can investigate whether the person has left the coverage area from his/her movement track, shown as [22] in FIG. 10 or there has been a failure of the monitoring device. Again, through two-way communication or resident local response personnel, the problem can be quickly resolved.

Another feature of the system is incorporating a simple keypad on the monitoring device, shown as [5 and 6] in FIG. 1, to allow a manual activation of an emergency alert (including personal security). Entry of normal, high or low measurement index on some physiological measurement that have to be done manually, such as determining the blood sugar level via pricking one's finger with a blood glucose meter. This keypad with a special key stroke sequence can also be used to temporarily suspend the alert threshold level by the person under monitoring prior to vigorous exercise or sexual activities or to countermand an issued alert. This approach drastically reduces or eliminates false alarms, thus rendering this emergency alert system much more effective.

To expand the freedom of movement, another feature of the system is integrating a modified relaying transceiver to a cell phone that a person typically carries. This device will be activated by a special signal emitted by the first tier relaying transceivers located on the perimeter of the coverage area, and it will relay the monitoring report and alert through the cell phone network to the remote monitoring center instead of the Internet. The cell phone tower position provides the added fix on the individual's location along with a built-in GPS receiver of the monitoring device.

We claim:

1. A transparent and automated personal medical emergency alert system consists of:
(1) a wearable monitoring device contains data processing and controlling as well as radio frequency transceiver/antenna circuitries which are integrated with sensors within a single or multiple enclosures to measure, on a periodic basis, a person's physiological parameters, such as pulse rate, heart rate, EKG, blood pressure, blood oxygen level, breathing rate and/or body temperature. It will compare the measurements against the threshold levels stored or pre-programmed in its memory to determine whether abnormality or severe abnormality has occurred and will wirelessly transmit via radio frequency (RF) signals corresponding to medical emergency alert, abnormal readings or normal measurements along with its unique identification code. Each monitoring device (consequently each person wearing this device) will be assigned a unique identification code, and it will not process or react to any incoming RF signals without its unique identification code as part of signals. This wearable monitoring mean is also integrated with an impact sensor for detecting a severe fall as well as a microphone to detect loud scream to issue an emergency alert for assistance. Also included in this wearable monitoring mean is a simple keypad for manually issuing emergency alert, temporarily suspending measurements and for manually entering ranges of blood glucose measurement (high, normal or low) after being prompted (by built-in vibration and/or audio component) periodically to conduct the measurement with a blood glucose meter.
(2) a battery charger that can rapidly charge up the built-in battery of the wearable monitoring device, a spare battery or a spare wearable monitoring device with same identification code to allow un-interrupted monitoring.
(3) a network of distributed wireless RF relay transceivers which will receive the signals from the wearable monitoring devices, determine the strength of each received signal and retransmit the signal along with its own unique identification code and signal strength data (thus providing the location data to the remote monitoring center) at a different frequency and power level (maybe utilizing a separate antenna tuned and oriented for long distance transmission) to another relaying transceiver for furthering distance of transmission or an Internet gateway directly.
(4) an Internet gateway which will convert the received RF signals from the distributed relay transceivers into proper protocol (such as TCP/IP) and transmit the data to a remote monitoring center via the Internet or an intranet.
(5) a remote monitoring center will process and archive the data received from various gateways according to the following software routines:
(i) if an emergency alert is received, it will flash in a prominent alarm color (such as red) the person's identification, calculated location (derived from the signal strength, position of the reporting relays and historical movement track of the subject, or from the Global Positioning Satellite receiver incorporated within a wearable monitoring device), type of medical emergency and the information corresponding local emergency response team on the monitoring screen for the monitoring personnel to react immediately. The remote monitoring center will also continuously forward the physiological measurement data and medical history of the person in need of assistance to the local response team;
(ii) if abnormalities in physiological measurements are received, it will flash in a less alarming color (such as amber) the person's identification, calculated location, type of medical abnormalities and the corresponding local resident response personnel information on the monitoring screen for the monitoring personnel to respond immediately;
(iii) if normal measurements are received, it will simply calculate location and archive the data without cautionary display on the monitoring screen.
(6) a local emergency response team which consists of at least one contracted ambulatory firm and a resident response personnel (a nurse, nurse-aid or a medical technologist). This team will respond to the direction of the remote monitoring center to render immediate assistance to the person in need.

2. The automated emergency alert threshold as described in claim 1 can be pre-programmed into a wearable monitoring device based on the physical conditions (such as age, gender, ethnicity, weight and special medical conditions) of the subject being monitored, or it can be derived from a general set of criteria based on subjects of similar age, gender, ethnicity, weight and normal medical conditions while adjusted automatically by the software residing in the remote monitoring center based on the average of measurements after a period (such as a few days) of continuous monitoring or after a history of measurements can be established. The threshold levels can also be manually adjusted by the subject or his/her physician (through either keypad of the wearable sensor device or through the Internet access) to accommodate prescription of new medication, treatment or new circumstances.

3. The automated emergency alert threshold as described in claim 1 can be further adjusted corresponding to typical activity levels of the subject throughout a day.

4. The automated emergency alert threshold as described in claim 1 can also be temporarily suspended by the subject under monitoring using the keypad on the monitoring device through a sequence of key strokes prior to exercises or extraneous activities. The key stroke sequence can also be used to cancel an alarm.

5. The system described in claim 1 can further utilize radio frequencies designated by the U.S. Federal Communication Commission for short distance communication without special licensing, such as 2.4 GHz, 315 MHz, etc.

6. The system described in claim 1 can further have its relaying transceivers distributed to provide redundancy in coverage area to allow each reporting signal from a wearable monitoring device to be received by at least two or more relaying transceivers to ascertain that each reporting signal will be properly relayed to the monitoring center. Each relaying transceiver will be powered by a combination of a rechargeable battery and a switching AC/DC power source to assure its functionality at all time.

7. To avoid mis-identification of subject and his/her medical status, the relaying transceivers described in claim 1 can further employ a hand-shaking method of having the monitoring device first transmit its identification code for a period of a few seconds at a specific frequency rate (such as once every second) when reporting until one or more relay transceivers responds by a “O.K. send report” signal in conjunction with this monitoring device ID code. The monitoring device will then transmit its data string repeatedly in burst mode (such as 10-50 microsecond burst) until all responding relaying transceivers each sends a “received” signal back (again including the ID code of the monitoring device). This hand-shaking method assures that each reporting signal strings are received properly by one or more relaying transceiver, matched with monitoring device ID code and forwarded to the remote monitoring center accurately.

8. The medical emergency alert system described in claim 1 can further have multiple tiers of relaying transceiving devices. Each relaying transceiver will have two separate antenna tuned to transmit and receive at two different frequencies. Whereas the first tier devices will be tune to receive signals from wearable monitoring devices (such as at 2.4 GHz) and to transmit at a different frequency (such as 315 MHz) to a second tier relaying transceiver, which, in turn, is tuned to receive at this frequency (e.g. 315 MHz) and transmit at another frequency.

9. The relaying transceiver described in claim 1 can further have a unique identification code assigned to each, and it will employ the following hand-shaking method to assure accurate signal transmission and avoid interference from other transmitting devices: it will first transmit its identification code for a period of a few seconds at a specific frequency rate (such as once every second) when relaying a set of signals received from one or more monitoring devices until a next tier relay transceivers responded with a “O.K. send report” signal in conjunction with the ID code of this first tier relaying transceiver. Then, it will transmits its data strings repeatedly in burst mode (such as 10-50 microsecond burst) till the responding next tier relay transceivers sends a “received” signal back (again including the ID code of the relaying transceiver device). This hand-shake method will be repeated from one tier to the next in relaying the data received from the monitoring device on either direction of communication.

10. The wearable monitoring device described in claim 1 can be in the form of wrist band, integrated with a watch module to form a wrist watch, an arm band, a leg band, a head band or a torso belt. Its sensors, processing and transceiving circuitry along with display, keypad and rechargeable battery will be enclosed in a waterproof structure. Furthermore, its can have an inscription of the name of the subject along with key medical information and contact phone numbers on its exterior.

11. The wearable monitoring device described in claim 1 can further include a battery level monitoring circuitry along with audio, video and/or vibration prompts to alert the person wearing the device to recharge the battery as well as transmitting a code for the remote monitoring center to remind the subject of this impending malfunction.

12. The wearable monitoring device described in claim 1 can further be connected (wirelessly or wired) to separate sensor pads (in addition to the sensors integrated within), such as transducers for recording electrocardiogram (EKG), for measuring breathing rate and/or blood pressure sensor. The measurements received will be transmitted periodical in conjunction with those derived from the integrated sensors. These separated sensor pads can be in disposable format or equipped with a rechargeable battery for reuse.

13. The wearable monitoring device described in claim 1 can also be programmed to periodically remind (such as by audio, video and/or vibration prompts) a diabetic subject to perform a manual blood glucose measurement with a glucose meter and enter the data on the keypad of the wearable device for transmission. The prompts will be repeated until a data entry act has been done.

14. The wearable monitoring device described in claim 1 can further include a speaker in conjunction with a built-in microphone to achieve two way verbal communications with the remote monitoring center and/or the emergency response personnel. The verbal communications will be digitized into a signal string and incorporate the unique identification code of a monitoring device (thus, the subject). The wearable monitoring device will only decode any incoming signals incorporated with its unique identification code, thus achieving exclusivity in two way verbal or text communications.

15. The wearable monitoring device described in claim 1 can further include a GPS receiver to determine its location and provide redundant position data for the remote monitoring center.

16. The method in determining the location of a subject issuing emergency alert as described in claim 1 can also be a combination of using signal strength received by the overlapping relaying transceivers at fixed locations (the intersection of two or more circles of different radii from each relaying transceiver, which signify different signal strengths) and movement track of the subject from his/her immediate past periodic reports.

17. The critical malfunctions for the system described in claim 1 can be diagnosed by determining whether there are periodic reporting signals from a specific wearable monitoring device or the reporting signal provides no physiological measurement data. In the event of no signal, the software resided at the remote monitoring center will examine the past reports from the device in question to see whether it is due to battery drain (continuous decline in signal strength or receiving battery drain signal from the monitoring device), moving out of the coverage area (movement track of the subject) or device failure (sudden cease of signal). In the case of no physiological measurement data, again the prior reports will indicate whether it is due to sudden device failure or simply the subject no longer wearing the monitoring device. The remote monitoring center will notify the local resident emergency response personnel about the type of malfunction and to contact the subject to resolve the problem, which can be simply replacing the wearable monitoring device with a spare device having the same identification code or change the battery.

18. The remote monitoring center described in claim 1 can further include software and/or resident physicians to provide likely medical diagnosis based on received physiological measurements to assist the local emergency response team in giving timely and proper treatment.

19. A relaying transceiver described in claim 1 can be in the form of an integrated component within a cellular phone to relay the physiological measurements and emergency alert signals to the remote monitoring center via cell phone networks. This relaying transceiver can be activated automatically by a special signal code, which the perimeter relaying transceivers of a coverage area transmits continuously, so when a subject is departing the coverage area, his/her physiological measurement and emergency alert signals can be received by the remote monitoring center without interruption. Furthermore, the cellular network will be able to provide location information (by triangulation) as part of the alert information package in addition to the GPS location data transmitted by the wearable monitoring device. This approach will further expand the coverage area of the system (thus freedom of movement of the subjects under monitoring) described in claim 1 to wherever cell phone signals can be received.

20. The employment of the system described in claim 1, as a paid service, can be subscribed by individuals, a community or by an entity such as a recreation and entertainment facility to provide medical emergency alert and security coverage.

21. The employment of the system described in claim 1 as an integral part of service for a retirement community, a mental hospital and/or nursing care facility to permit its residents or patients freedom of movement while providing continuous medical monitoring and emergency alert response.

22. The system described in claim 1 can also be linked to a home security and/or fire alarm monitoring system to provide a comprehensive safety monitoring for an individual/family.

23. Utilizing pre-arranged security codes (such as passwords), relatives, police, or legal guardians of a person suffering Alzheimer's or dementia can use the system described in claim 1 to monitor, locate and track the movement of the subject via the Internet.

24. Utilizing pre-arranged security codes (such as passwords), personal physicians of a subject person under monitoring by the system described in claim 1 can access the archived physiological measurements via the Internet to track the wellness of the person, particularly after new medication or treatment is instituted.
